SEQUENCE LISTING (5 sequences)

SEQ ID NO: 1 - DNA, 19 bp, Artificial Sequence
  Sequence targeted for SERCA1b silencing
ctatctggaggatccagaa

SEQ ID NO: 2 - DNA, 63 bp, Artificial Sequence
  Oligo to obtain transcription of interfering RNA
gatccgctatctggaggatccagaattcaagagattctggatcctccagatagtttttggaaa

SEQ ID NO: 3 - DNA, 63 bp, Artificial Sequence
  Oligo to obtain transcription of interfering RNA
agcttttccaaaaactatctggaggatccagaatctcttgaattctggatcctccagatagcg

SEQ ID NO: 4 - DNA, 20 bp, Artificial Sequence
  Primer for amplification of EGFP coding sequence
acgtaaacggccacaagttc

SEQ ID NO: 5 - DNA, 20 bp, Artificial Sequence
  Primer for amplification of EGFP coding sequence
aagtcgtgctgcttcatgtg

(19) United States
(12) Patent Application Publication
(10) Pub. No.: US 2011/0313027 A1
(43) Pub. Date: Dec. 22, 2011

(54) METHODS AND SUBSTANCES FOR STIMULATING MUSCLE REGENERATION

(76) Inventor: Ernö Zádor, Szeged (HU)

(21) Appl. No.: 13/058,581

(22) PCT Filed: Aug. 13, 2009
(86) PCT No.: PCT/HU09/00077

§ 371 Date: Feb. 13, 2011

(30) Foreign Application Priority Data
Aug. 13, 2008 (HU) P0800515

(51) Int. Cl. A61K 48/00; C12Q 1/68; A61P 21/00; C12N 15/63

(52) U.S. Cl. 514/44 R; 435/320.1; 435/6.13

(57) ABSTRACT

The invention is concerned with the field of muscle growth and regeneration. More particularly, the invention provides novel methods, substances and uses for facilitating the growth or regeneration of skeletal muscles. The methods, uses and substances of the invention are useful in the treatment or prevention of degenerative-regenerative muscular disorders or in the treatment of muscle injuries. The substances provided by the invention are capable of upregulating the calcineurin-NFAT-IL-4, advantageously by inhibiting the activity of the Ras or SERCA1b gene product. Introduction of the substances provided by the invention into only a small percentage of the fibers present in a regenerating muscle facilitates the regeneration of the whole muscle concerned.

### FIELD OF THE INVENTION

The invention is concerned with the field of muscle growth and regeneration. More particularly, the invention provides novel methods, substances and uses for facilitating the growth or regeneration of skeletal muscles. The methods, uses and substances of the invention are useful in the treatment or prevention of degenerative-regenerative muscular disorders or in the treatment of muscle injuries. The substances provided by the invention are capable of upregulating the calcineurin-NFAT-IL-4, advantageously by inhibiting the activity of the Ras or SERCA1b gene product. Introduction of the substances provided by the invention into only a small percentage of the fibers present in a regenerating muscle facilitates the regeneration of the whole muscle concerned.

### ABBREVIATIONS USED

NFAT—nuclear factor of activated T-cells; IGF-I—insulinlike growth factor I; IL-4—interleukin-4; EGFP—eukaryotic green fluorescent protein; IL-4—interleukin-4; MyHC1—slow type myosin heavy chain; NFAT—nuclear factor of activated T-cells; RNAi—RNA-interference; RT PCR—reverse transcriptase polymerase chain reaction; SERCA1a—adult fast type sarcoplasmic/endoplasmic reticulum Ca²⁺ ATPase; SERCA1b—neonatal sarcoplasmic/endoplasmic reticulum Ca²⁺ ATPase; SR—sarcoplasmic reticulum.

### BACKGROUND OF THE INVENTION

Skeletal muscle has a remarkable potential to regenerate if the innervation, the vascularization and the connective tissue remain relatively intact [1]. The effect of innervation is mediated by two distinct pathways in regenerating slow-twitch soleus muscle, one is the calcineurin-NFAT and the other involves Ras (reviewed in [2]). In spite of their parallel function, little information is available about the in vivo connection of calcineurin and Ras in skeletal muscle.

Calcineurin is activated by the Ca-microdomains, then it dephosphorylates the NFAT isoforms and stimulates their transport to the nucleus (reviewed in [3]). In skeletal muscle NFAT acts as a nerve activity sensor [4]. The nuclear accumulation of NFATc1 helps to gain slow fiber type identity [5] while NFATc2 stimulates growth of myotubes and maturing myofibers [6,7]. The NFATc2 upregulates the transcription of the interleukin-4 (IL-4) gene. The secreted IL-4 binds to receptors on the surface of myoblasts and enhances the recruition of myoblasts into the fibers. Skeletal muscle fibers grow by accretion of myoblasts. Therefore the calcineurin-NFATc2-IL-4 pathway is an important regulator of autocrine/paracrine growth of muscle fibers [8]. The activity of calcineurin is increased by innervation in regenerating muscle [9,10] suggesting a nerve dependence of this factor upon differentiation of slow muscle type.

Ras (a well known small GTPase being involved in cellular signal transduction) also mediates the effect of innervation in slow muscle differentiation. One of the downstream effector of Ras is the phosphatidylinositol-3-OH kinase (PI3K) [11]. The PI3K upregulates the Akt/PKB-mTor-S6K pathway which increases the protein synthesis in the ribosomes of regenerating and normal muscles [12,13]. Therefore Ras stimulates muscle growth in a fiber-autonomous fashion, while the calcineurin-NFATc2-IL-4 pathway increases muscle growth in autocrine-paracrine manner [8].

In contrast to the reported role of Ras in fiber development [1]-13], the size of the fibers transfected with dominant negative Ras (dnRas) is not decreased compared to the untransfected fibers in the regenerating soleus muscle [11]. This suggests that the growth of the dnRas transfected and the untransfected fibers are somehow synchronized. Such a mechanism would require secretable factors which, according to an analysis of the human genome, are expressed in a vast number in skeletal muscle [14]. Ras and calcineurin are members of two independent pathways in muscle growth and their interaction is not known.

In order to enlighten this problem, regenerating soleus muscles of the rat were transfected with dnRas and studied for growth. Furthermore, the present inventor has also tested the effect of the activation of the calcineurin-NFAT-IL-4 pathway on regenerating skeletal muscle via another way, namely by silencing the SERCA1b gene. The neonatal isoform of the sarcoplasmic/endoplasmic reticulum Ca2+ ATPase 1 (SERCA1b) represents a dominant Ca2+ pump in fibers of regenerating muscle.

Developing skeletal muscle fibers can grow by recruition of new myoblasts [42, 47]. The elevation of sarcoplasmic Ca2+ level in the fiber controls muscle growth by stimulating the calmodulin dependent calcineurin-NFAT pathway [34, 6]. Calcineurin dephosphorylates NFAT, which thereupon translocates to the nucleus, and increases, amongst others, the expression of interleukin-4, a documented autocrine and paracrine factor of muscle fiber growth. The secreted interleukin-4 binds to its receptors on the membrane of myoblasts and stimulates their accretion to the fibers [8]. The sarcoplasmic/endoplasmic reticulum Ca2+ ATPases (SERCAs) represent the main cellular Ca2+ pumps mediating the decrease of the sarcoplasmic level of Ca2+ by reaccumulating the ions into the sarcoplasmic reticulum.

The inhibition of SERCAs by thapsigargin decreases myotube growth and differentiation [49, 50]. When elaborating the present invention, I have focused on the neonatal isoform SERCA1b [36, 35, 43] in an effort to elaborate further advantageous embodiments. The mRNA of SERCA1b is expressed in a regenerating fast-twitch and slow-twitch skeletal muscle [53, 55]. The cDNAs of SERCA1b and of the corresponding adult fast type muscle SERCA1a isoform have been expressed in COS cells and no differences were found in the maximal rates nor in the Ca2+ dependency of Ca2+ transport into the microsomes of these cells [44]. SERCA1 knockout mice, which lack both SERCA1a and SERCA1b, die shortly after birth in gasping respiration and cyanosis, probably because the hypercontracted diaphragm is not able to support breathing [48]. However, human Brody disease (i.e. a rare inherited disorder associated with a defective SERCA1 function) patients show a much milder phenotype, only characterized by exercise-induced impairment of skeletal-muscle relaxation, stiffness and cramps, but without the lethal respiratory muscle defect [37]. These data leaves the question why this neonatal isoform is expressed unanswered. Earlier, the present inventor has found the SERCA1b protein being expressed specifically in early developing fibers of in vivo regenerating muscles [55].

### SUMMARY OF THE INVENTION

The present inventor has surprisingly found that the transfection of about 1% of the muscle fibers with dominant negative Ras (dnRas) shows a wilder effect; it stimulates the fiber growth in the entire regenerating soleus muscle, including the nontransfected fibers. The expression of dnRas in only a few fibers increased the mass and fiber size in the whole regenerating soleus muscle until day 21 of regeneration. Cotransfection with the calcineurin inhibitor cain/cabin prevented the growth stimulation. Injection of antibody for interleukin-4 (IL-4) also abolished the growth ameliorating effect. These results suggest that the inactivation of Ras in 1% of the fibers upregulates the calcineurin-NFAT-IL-4 pathway and the secreted IL-4 triggers fiber growth stimulation in the whole regenerating soleus muscle of the rat. The results highlight the importance of the autocrine-paracrine regulation in muscle regeneration and hint to a novel method of gene therapy of degenerative-regenerative muscle dystrophies.

Furthermore, the present inventor has also found that, surprisingly, silencing of SERCA1b in only a few new fibers, as a result of injecting a regenerating soleus muscle with a SERCA1b-directed RNAi plasmid, showed a widespread effect. It induced an increase of fresh weight and the fiber size in the whole muscle, including the nontransfected fibers. Coinjection into the same fibers, but not the separate injection into different fibers of vectors expressing the calcineurin inhibitor domain of cain [19] and the SERCA1b RNAi, neutralized the growth-promoting effect and even reduced muscle growth. Notably, the growth-promoting effect of SERCA1b RNAi was also abolished by perimuscular injection of interleukin-4 (IL-4) antibody. This indicates that SERCA1b affects calcineurin activity and thereby controls the interleukin-4 stimulated muscle growth in regeneration.

### DETAILED DESCRIPTION OF THE INVENTION

In accordance with the above summarized unexpected findings, the present invention provides for substances capable of upregulating the calcineurin-NFAT-IL-4 pathway in organisms having skeletal muscle for use in facilitating the growth or regeneration of skeletal muscle, in the treatment or prevention of degenerative-regenerative muscular disorders and in the treatment of muscle injuries. The treatment or prevention methods of the invention advantageously include gene therapy

The invention further concerns the use of a substance capable of upregulating the calcineurin-NFAT-IL-4 pathway in an organism having skeletal muscle for the preparation of a pharmaceutical composition for the treatment or prevention of degenerative-regenerative muscular disorders, facilitating the growth or regeneration of a skeletal muscle or for the treatment of muscle injuries in said organism.

Substances applicable in accordance with the present invention are advantageously capable of upregulating the calcineurin-NFAT-IL-4 pathway by inhibiting the activity of the Ras or SERCA1b gene product, e.g. by inhibiting the expression of said gene products or by inhibiting the activity of the expressed gene products. Advantageous substances according to the invention are e.g. vectors enabling the expression of RNA or protein molecules capable of inhibiting the expression of the Ras or SERCA1b gene or the activity of the Ras or SERCA1b gene product in skeletal muscle cells. Such advantageous embodiments of the invention are e.g. vectors encoding dominant negative Ras (dnRas) or vectors capable of expressing a SERCA1b-RNAi. The person skilled in the pertinent art will comprehend that the calcineurin-NFAT-IL-4 pathway may be triggered via other routes and by using other substances than those specifically exemplified herein. Such other substances may be easily identified by a person skilled in the pertinent art without undue burden on the basis of the present disclosure and, therefore, such substances are considered as being covered by the scope of the present invention.

The treatment in accordance with the invention is advantageously is made by introducing a substance according to the invention only into a part of the muscle fibers present in a skeletal muscle to be treated, advantageously into about 0.1-10%, about 0.2-5%, about 0.5-2% or about 1% of the muscle fibers present in said skeletal muscle, whereby said growth or regeneration facilitating effect induced will be detectable both in fibers of said muscle where said substance was introduced and in fibers where it was not.

Organisms that can be treated in accordance with the invention are advantageously vertebrates, more advantageously mammals and even more advantageously humans.

The invention also provides a method for identifying a substance useful for facilitating the growth or regeneration of skeletal muscle, or the treatment or prevention of degenerative-regenerative muscular disorders, or the treatment of muscle injuries comprising the steps of

i) providing a cell or tissue culture or a model animal in which the activity of calcineurin is experimentally assessable;

ii) contacting a test substance with said cell or tissue culture or model animal;

iii) assessing the activity of calcineurin in said cell or tissue culture or model animal after contacting the same with said test substance; and

iv) identifying said test substance as a substance useful in the treatment or prevention of degenerative-regenerative muscular disorders or in the treatment of muscle injuries if the calcineurin activity assessed in step iii) is significantly higher than the calcineurin activity assessable in said cell or tissue culture or model animal before said contacting with said test substance performed in step ii).

The invention further provides a method for identifying a substance useful for facilitating the growth or regeneration of skeletal muscle, or the treatment or prevention of degenerative-regenerative muscular disorders, or the treatment of muscle injuries comprising the steps of

i) providing a cell or tissue culture or a model animal in which the activity of the Ras or SERCA1b gene is experimentally assessable;

ii) contacting a test substance with said cell or tissue culture or model animal;

iii) assessing the activity of the Ras or SERCA1b gene in said cell or tissue culture or model animal after contacting the same with said test substance; and

iv) identifying said test substance as a substance useful in the treatment or prevention of degenerative-regenerative muscular disorders or in the treatment of muscle injuries if the Ras or SERCA1b activity assessed in step iii) is significantly lower than the Ras or SERCA1b activity assessable in said cell or tissue culture or model animal before said contacting with said test substance performed in step ii).

The invention further provides methods for facilitating the growth or regeneration of skeletal muscle, or treating or preventing degenerative-regenerative muscular disorders, or treating muscle injuries comprising introducing a substance capable of upregulating the calcineurin-NFAT-IL-4 pathway into said skeletal muscle, said substance being advantageously capable of inhibiting the activity of the Ras or SERCA1b gene product. In accordance with the treatment method according to the invention, said introduction is advantageously made only into a part of the muscle fibers present in a skeletal muscle to be treated, advantageously into about 0.1-10%, about 0.2-5%, about 0.5-2% or about 1% of the muscle fibers present in said skeletal muscle, whereby said growth or regeneration facilitating effect induced will be detectable both in fibers of said muscle where said substance was introduced and in fibers where it was not. The substances useful in the treatment methods of the invention are advantageously substances capable of inhibiting the activity of the Ras or SERCA1b gene, being advantageously vectors enabling the expression of an RNA or protein molecule capable of inhibiting the expression of the Ras or SERCA1b gene or the activity of the Ras or SERCA1b gene product in skeletal muscle cells, advantageously vectors encoding dominant negative Ras (dnRas) or vectors capable of expressing a SERCA1b-RNAi.

### BRIEF DESCRIPTION OF THE DRAWINGS

FIG. 1: The transfection with dnRas increases fresh weight and fiber size in the whole regenerating soleus muscle. The increase of (A) fresh weight and (B) fiber size of the regenerating soleus after transfection of a few muscle fibers with plasmid expressing dominant negative Ras (RasN17). ** p<0.01, *** p<0.001 compared to regenerating muscles transfected with the empty vector, pRSVlacz; ++p<0.01, +++ p<0.001 compared to the 12 days regenerating muscles transfected with the same plasmids. The identification of (C) the pRSV lacz transfected fibers with beta-galactosidase staining and (D) the pRSV RasN17 expressing fibers with immunostaining by pan Ras antibody. Bar indicates 200 μm.

FIG. 2: Co-transfection of cain/cabin and dnRas into the same fibers prevents the increase of (A) muscle weight and (B) fiber size in the whole regenerating soleus. The transfection of dnRas and cain/cabin into separate fibers of the same muscle did not prevent the dnRas-stimulated growth. Vector—the empty plasmid used for expression of cain/cabin. + means co-transfection and ( ) indicates the subsequent transfection of two plasmids into the same muscle. * p<0.05; *** p<0.001 compared to the muscles transfected with the empty vector of cain/cabin. Representative immunograph of parallel sections stained for (C) dnRas and (D) the myc-tag of cain/cabin in the same fibers. (E) Ras-staining and (F) slow myosin heavy chain (MyHCI) staining of parallel sections of regenerating soleus muscles after subsequent transfection with dnRas and cain/cabin. On (F) the stars indicate fibers which express dnRas on (E). The MyHC1-negative fibers reflect the transfection either with dnRas or cain/cabin, since both dnRas and cain/cabin prevents the expression of slow MyHC1. Note that more MyHC1-fibers are present on (F) than Ras+ fibers on (E) indicating in addition the cain transfected fibers. Bar indicates 100 μm.

FIG. 3: The phenotypical appearance of the transfected muscles. The following symbols indicate the transfecting plasmids: RSV—pRSVlacz, dnRas.-.pRSV RasN17, dnRas+cain—pRSVRasN17+ cain, 28d—28 days regenerating soleus, N—normal soleus

FIG. 4: The dnRas stimulated muscle fiber growth is mediated by interleukin-4. (A) The immunoblot of IL-4 from 3-3 dnRas transfected and pRSV lacz (vector) transfected regenerating soleus muscles. The representative immunograph of the fibers transfected by dnRas (B) secreting more IL-4 (C) than the untransfected fibers. Fibers transfected with the control pRSV lacz (D) do not express more IL-4 (E) than the untransfected fibers in the same muscle. Stars indicate identical positions on parallel sections. (F) Antibody to interleukin-4 prevents the autocrine-paracrine stimulation of fiber growth in dnRas transfected regenerating soleus muscles. A-IL-4—anti-interleukin-4, IgG—irrelevant mouse IgG. *** p<0.001 compared to the other column. Bar indicates 100 μm.

FIG. 5: SERCA1b is expressed in developing fibers of regenerating muscle. (A) The neonatal SERCA1b in normal (N) and 3-28 days regenerating soleus [29]. The comparison of SERCA1b and SERCA1 levels in the SR fraction of day 7 (B) and day 21 (C) regenerating muscles. Note that corresponding bands on day 7 stained with a similar intensity with both the SERCA1 and the SERCA1b antibodies showing that SERCA1b is the main SERCA1 isoform whereas SERCA1a is nearly absent in that stage. Meanwhile on day 21 the SERCA1 signal is apparently shared between SERCA1a and SERCA1b. The inset shows examples of immunoblots. N=3, the vertical bars mean standard errors, * p<0.05, ** p<0.01 SERCA1b vs. SERCA1

FIG. 6: RNAi silencing of SERCA1b expression in COS-1 cells. Cotransfection of the SERCA1b RNAi and the SERCA1b expressing plasmids into COS-1 cells in 48:1 molar ratio decreased the SERCA1b mRNA level with nearly 70% of that of the transfection control (empty vector). The mismatch RNAi did not show an inhibitory effect. The PCR amplifications were made in the linear range for both SERCA1b and EGFP as depicted in the insert which shows the individual bands of the amplifications. ** p<0.01 with respect to vector, +p<0.05 with respect to mismatch control.

FIG. 7: The RNAi inhibition of SERCA1b in a few fibers of regenerating soleus. (A) The fresh weight and (B) fiber cross sectional area in the whole regenerating soleus. Seven-days-regenerated soleus muscle injected (C-E) with RNAi construct or (F-H) with the empty vector. Arrows point to identical positions on parallel sections. The transfected fibers were identified by the expression of EGFP (C, F). Note that some of the EGFP-expressing fibers in RNAi treated muscle lack SERCA1b expression (D), also confirmed with the SERCA1 antibody (E). SERCA1b RNAi—RNAi construct; vector—empty vector; mismatch RNAi—a mutant RNAi construct used as control, ***—highly significant (p<0.001) and *—significant (p<0.05) difference from the control transfected regenerating muscle. Bar on H shows 50 μm.

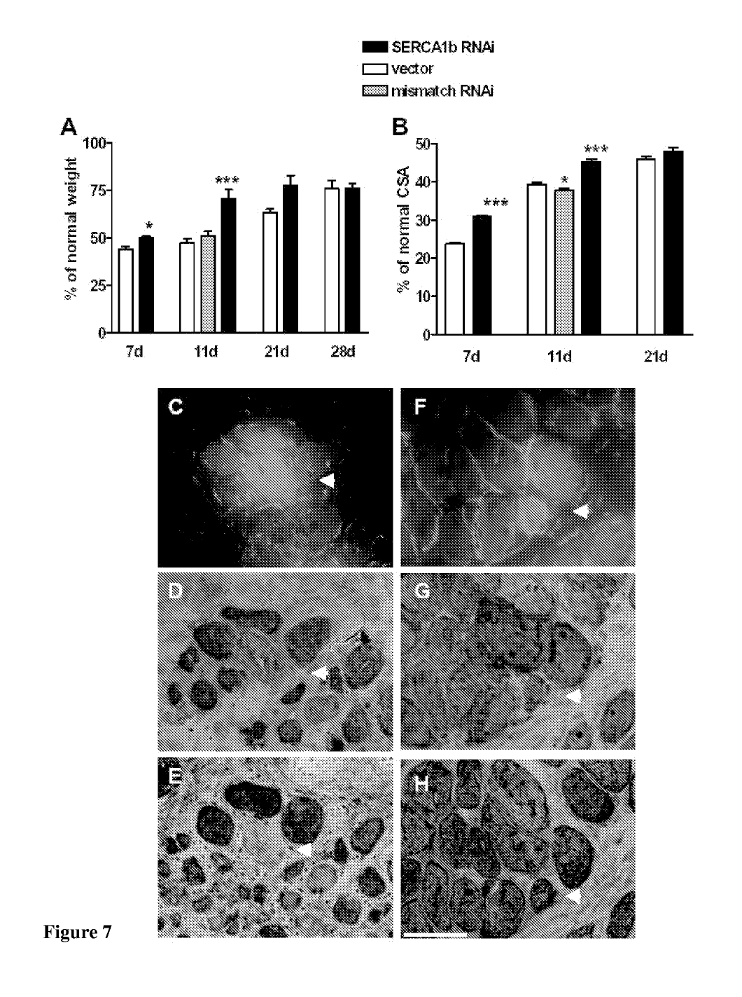

FIG. 8: Cotransfection of cain with SERCA1b RNAi prevents growth stimulation in muscle regeneration. (A) Fresh weight and (B) mean fiber size of cotransfected an subsequently transfected 12-days-regenerating muscles. Fibers transfected with cain and SERCA1b RNAi are identified by both the expression of EGFP (C) and the lack of MyHC1 (D) on parallel sections. Note that in the separately transfected muscles, fibers expressing EGFP (E) also express MyHC1 (F), whereas the MyHC1-negative fibers (F) do not express EGFP (E). RNAi+CV—SERCA1b RNAi cotransfected with the empty vector of cain; cain+RNAi—cain cotransfected with SERCA1b RNAi; cain/RNAi—cain and SERCA1b RNAi subsequently transfected with separate injections. Symbols as in FIG. 3. Stars on C-F indicate identical positions on parallel sections. Bar on F shows 50 μm.

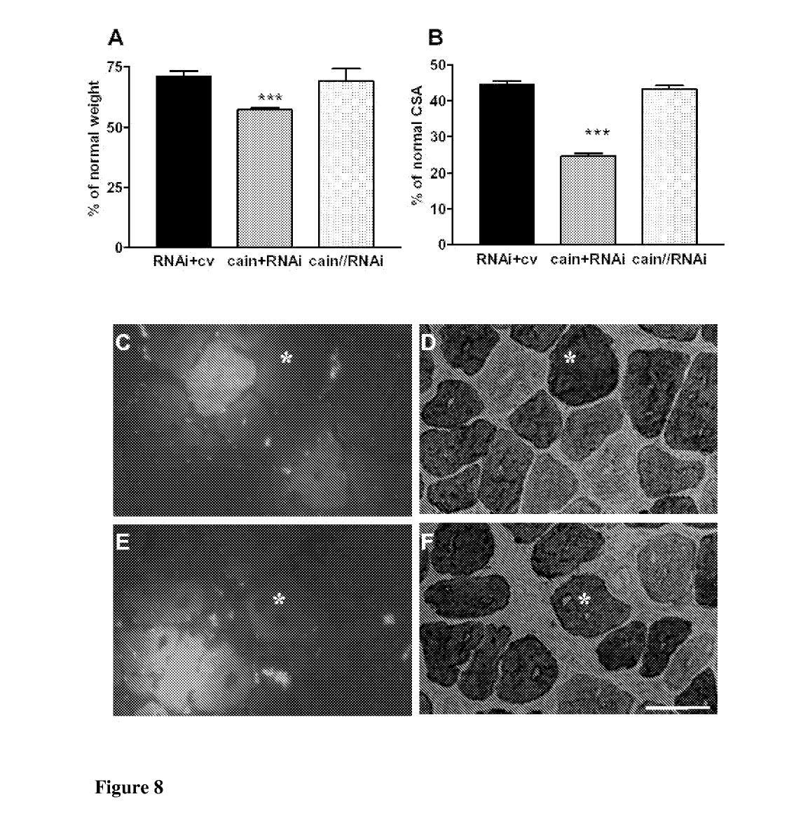

FIG. 9: The phenotypical appearance of the transfected regenerating muscles. 11 d and 28 d—nontransfected muscles regenerated for different lengths of time indicated as number of days. 11-days-regenerating soleus muscles transfected with empty vector of RNAi (V) and SERCA1b RNAi (RNAi). 12-days-regenerating soleus muscle cotransfected (RNAi+cain) or separately transfected (RNAi/cain) with SERCA1b RNAi and cain. N—normal soleus of a four-months-old rat.

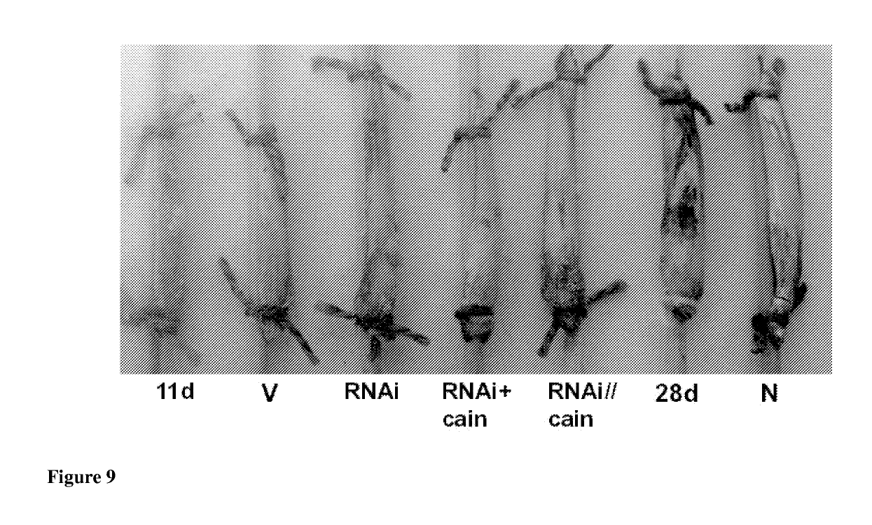

FIG. 10: The SERCA1b RNAi stimulated growth depends on interleukin-4. The results are shown at 11 days of regeneration. (A) SERCA1b RNAi increases the number of myonuclei. Nontransfected regenerating (reg) and regenerating soleus transfected with the empty RNAi vector (vector), the mismatch control (D3137) and the SERCA1b-RNAi expressing-vector (1bRNAi). The inset photograph (a colored version is also filed with the present application for information) shows a sarcolemma immunostaining with α-sarcoglycan and the DAPI-staining of nuclei. (B) Black columns show the effect of treatment with interleukin-4 antibody on fiber cross-sectional area in regenerating muscles transfected with SERCA1b RNAi (A-IL1bRNAi) or in muscles transfected with an empty control vector (A-IL vector); white columns represent the effect of treatment with unrelated antibodies of regenerating muscles transfected with SERCA1b RNAi (IgG1bRNAi) or empty vector (IgG vector). (C) Immunoblot of extract from SERCA1b-RNAi-transfected muscles with anti-interleukin-4. N=3-5, *** indicates p<0.001 compared to the regenerating muscles transfected with empty vector and treated with anti interleukin-4 or unrelated antibodies.

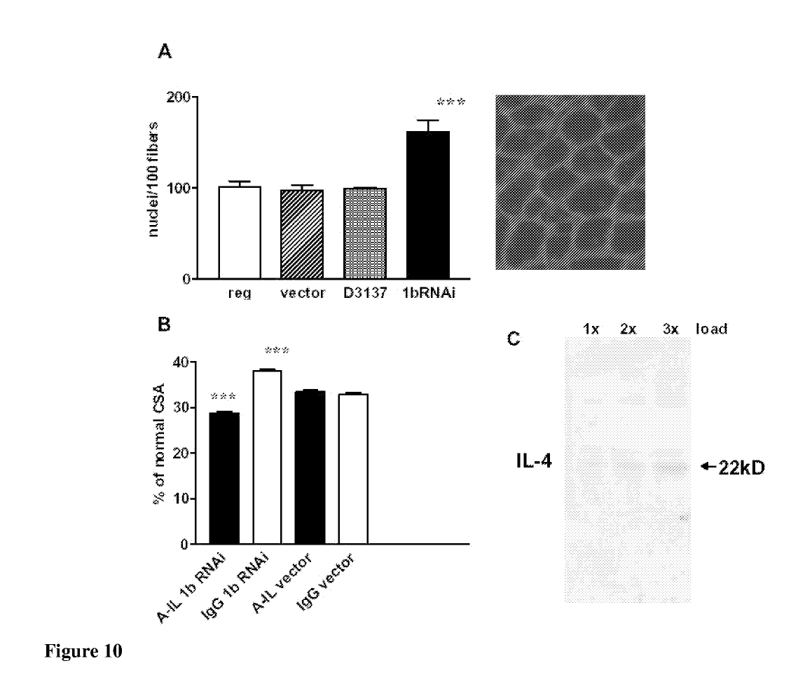

### EXAMPLES

The herein claimed invention will be further illustrated below by some disclosed experimental examples. The skilled person will understand that the below described experiments are only presented for exemplifying several advantageous embodiments of the invention and are not intended to be used as anyway limiting the claimed scope of the invention which, in turn, is solely defined by the appended claims. The person skilled in the pertinent art will certainly comprehend that many different modifications, alterations and substitutions by equivalent constituents or features may be performed with respect to the exemplified advantageous embodiments without significantly diverging from the spirit and scope of the claimed invention and, therefore, such modifications, alterations and substitutions remain within the claimed scope of the invention.

### Materials and Methods

Animal treatment, induction of regeneration, denervation, transfection of muscles. Experiments with animals were approved by the Ethical Committee of Animal Treatment of the Medical Faculty of the University of Szeged. Animals were treated according to the experimental ethic laws of the Medical Faculties at the University of Szeged and the Catholic University of Leuven.

The induction of regeneration in the soleus muscles of the left hindlimb of male rats (310-330 g) and muscle transfection after four days of regeneration was done as before [15,16], except that 30 μg of plasmid in 50 μl of 20% sucrose was transfected into the soleus and for co-transfection 30-30 μg of DNA of each plasmid was mixed. Subsequent injections with 15 min interval were made using 30-30 μg of DNA. Five μg of IL-4 antibody or irrelevant mouse IgG in 0.5 ml of 0.9% NaCl was injected from day seven of regeneration subcutaneously to the hindlimb of the dnRas transfected soleus. The muscles were dissected after 12 and 21 days of regeneration (8 and 17 days after plasmid transfection) and frozen in isopentane cooled in liquid nitrogen then kept at −70° C. until use.

Muscle necrosis was induced by the in situ injection of notexin, a snake venom [41] with some modifications [53] and the ensuing regeneration process in the soleus muscle was found to be remarkably reproducible as assessed by histological inspection and by a number of biochemical parameters [53, 54, 52, 51, 16, 55, 56]. Transfection with 30 μg of SERCA1b RNAi expressing pSuperEGFP vector in 50 μl of 20% sucrose on day four of regeneration was done as described previously [15]. In case of coinjection a mix of 25-25 μg of the cain-expressing vector [19] and the RNAi expressing plasmid was coinjected or separately injected as before [56]. 5 μg of interleukin-4 antibody (mouse, R&D Systems) or irrelevant mouse IgG was injected in 0.5 ml physiological salt subcutaneously into the left leg, near to the regenerating soleus from day seven of regeneration of the RNAi expressing or the empty vector transfected muscles.

Vectors and RNAi constructs. The pRSV RasN17 plasmid expressing the dominant negative mutant of H-Ras [17,18] was kindly provided by Dr. A. Serrano (Padua, Italy). The pRSV lacz plasmid was generously donated by Dr. E. Taparowsky. The plasmid expressing the calcineurin inhibitory domain of cain from CMV promoter [19, 20] was received from Dr. S. Schiaffino with permission of Dr. S. H. Snyder.

DNA templates for the synthesis of small interference RNA were cloned into the pSUPER-EGFP vector where they are under the control of an H1-RNA promoter. This vector was modified from pSUPER plasmid [38] by inserting the GFP cassette (enhanced GFP controlled by the CMV IE promoter and terminated by the SV40 polyadenylation signal) in order to directly visualize the efficiency of cell transfection. The GFP cassette was derived from the pEGFP-C1 vector (Clontech) and prepared as follows. pEGFP-C1 was cut with BglII/BamHI and then subsequently religated in order to remove the multiple cloning sites. The resulting plasmid was digested with AseI/MluI to generate the GFP cassette, which was blunt-ended and cloned into SmaI site in the pSUPER vector.

The sequence targeted for SERCA1b silencing was

5′-CTATCTGGAGGATCCAGAA-3′ (SEQ ID NO: 1),

corresponding to the last 11 bases of exon 21 (3135-3145b of the rat SERCA1 cDNA, acc. No. M99223) and the first 8 bases of exon 23 (3188-95b). These fragments are contiguous only in the spliced SERCA1b mRNA after skipping exon 22. To obtain transcription of interfering RNA targeting the SERCA1b sequence, I have designed the following oligos, in which the sense and the antisense sequences of the target are indicated in italics:

Top Strand:

| | |
|---|---|
| 5′-gatccgctatctggaggatccagaattcaagagattctggatcctccagatagtttttggaaa-3′; | (SEQ ID NO: 2) |

Bottom Strand:

| | |
|---|---|
| 5′-agcttttccaaaaactatctggaggatccagaatctcttgaattctggatcctccagatagcg-3′. | (SEQ ID NO: 3) |

The oligonucleotides were annealed and digested with BglII and HindIII and ligated together in the vector digested with the same enzymes, and then transformed into competent DH5-α cells. The vector, expressing the calcineurin inhibitory domain of cain fused to a myc epitope under control of a cytomegalovirus promoter [19], was digested with SalI and NotI to remove the inhibitory domain coding insert. The digest was blunt-ended with T4 DNA polymerase then ligated and transformed into competent cells. An empty vector was used as a control.

Immunoblot. The detection of interleukin-4 on immunoblot was done as in [10,15] using antirat IL-4 antibody (mouse, 1:500, R&D systems).

Immunostaining, fiber dimensions, counting myonuclei, statistics. The expression of dominant negative Ras was identified by the pan Ras (Ab-1) antibody (mouse 1:30; Oncogene™), the IL-4 was detected by the antibody (mouse 1:30, R&DSystems) and the expression of cain detecting the myc-tail by the anti-myc antibody (mouse 1:20, Sigma).

Frozen transversal sections (15 μm) of regenerating and normal soleus were immunostained for slow myosin heavy chain (MyHC1) as described previously [15]. For counting nuclei the α-sarcolipin antiserum (Novocastra) was applied in 1:100 and covered with Vectashield DAPI (Vector Laboratories). Fibers were measured and nuclei counted on whole cross sections taken from the middle part of the muscle using the Analysis program (Soft Imaging System GmbH, Münster, Germany) From each muscle the fiber cross sectional area was measured on three blindly chosen visual fields with the 10× objective. In each field >150 fibers were measured. Each set of experiments was repeated on 3-5 muscles. Nuclei were counted in 120-160 fibers per muscle. The Student's t-test and ANOVA Newman-Keuls post hoc test were used to evaluate the statistical significance or their deviation from the normal values.

Beta-galactosidase staining. Frozen sections were fixed in 2% paraformaldehyde of acetone for 5 min, rinsed 3× in PBS (pH7.5) and stained in X-gal buffer (100 mM PBS, 5 mM KFe(II)-cyanide, 5 mM KFe(III)-cyanide, 2 mM MgCl2, 1 mM X-gal) overnight at 37° C.

Fiber cross-sectional area. The Olympos DP-soft, version 3.2 program was used to measure cross-sectional area (CSA) of fibers on hematoxilin-eosin stained sections. 150 fibers were measured from each muscle.

Transfection into COS-1 cells. The pcDNA3.1 plasmid expressing SERCA1b with codons preferred in the yeast translation (kindly provided by Dr. Malcolm East) was made entirely homologous to the SERCA1b RNAi using the QuickChange® mutagenesis kit (Stratagene) and cotransfected with the SERCA1b RNAi expressing plasmid in 1:48 molar ratio into COS-1 cells with the GeneJuice™ method (Novagen). The transfected cells were harvested after two days and submitted to isolation of total RNA.

RNA isolation and RT PCR. Total RNA was isolated according to standard method [40]. The reverse transcription (RT) were done as described earlier [53, 51]. The primers and PCR conditions for SERCA1b were as in [53] for 18 cycles.

The primers for EGFP were

5′-acgtaaacggccacaagttc-3′ (SEQ ID NO: 4) and 5′-aagtcgtgctgcttcatgtg-3′ (SEQ ID NO: 5)

amplifying nt65-251 in cDNA of the inserted EGFP in the pSUPERB vector. The amplification was at 95, 50, 72° C. each for 1 min, for 14 cycles. The amplification of both SERCA1b and EGFP were adjusted in the linearity range.

Immunoblots. Crude fragmented SR membranes were prepared from muscles, subjected to SDS PAGE (70 μg of protein was loaded per lane), blotted onto PDVF membranes and processed for immunoblots as described previously [54, 51, 15]. SERCA1b-specific antiserum (rabbit, 1:2000) or monoclonal antibody A3 (mouse, 1:100 of culture supernatant; kindly provided by Dr. D. H. MacLennan, Toronto, Canada) which recognizes an epitope common to SERCA1a and SERCA1b [57] were used as 1st antibody.

### Example 1

### In Vivo Transfection of about 1% of the Fibers with Plasmid Expressing dnRas Stimulates the Fresh Weight and Fiber Size Regenerating Soleus Muscle in Rats

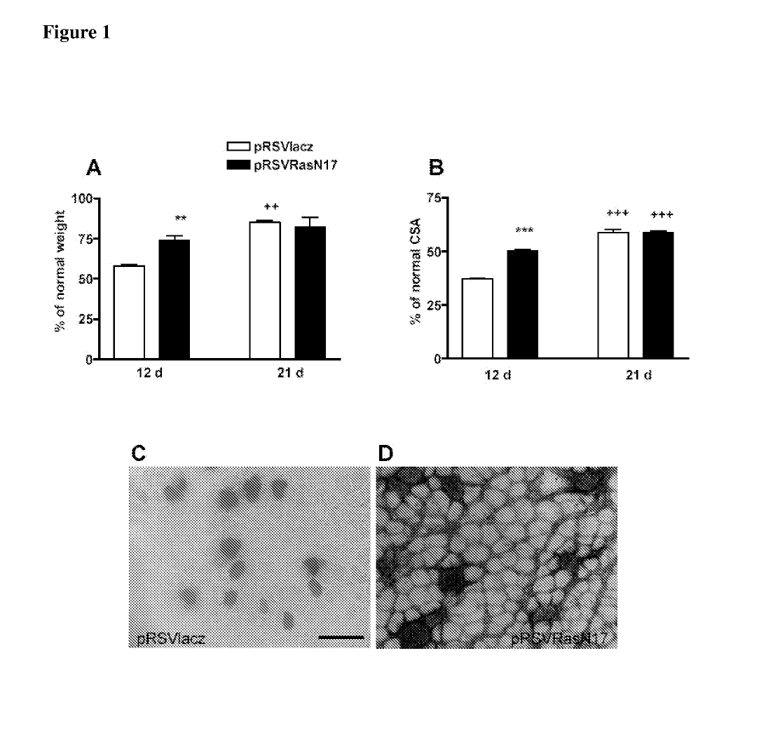

The regenerating soleus muscle gradually increased mass and fiber size to 98% and 83% of those of the normal soleus within 28 days following the notexin-induced necrosis (Table 1). The even progress of regeneration made feasible the detection of stimulation of fiber growth. In vivo transfection of about 1% of the fibers (i.e. 24 out of 2400 of total fibers [16]) with plasmid expressing dnRas stimulated the fresh weight and fiber size compared to the control regenerating muscles transfected with the pRSV lacz vector at 12 days of regeneration in the soleus (FIG. 1AB). The dnRas transfected fibers were identified by the pan Ras antibody and the pRSV lacz transfected fibers by the expression of beta-galactosidase (FIG. 1CD). The dnRas expressing fibers did not show any difference in size compared to the untransfected fibers in the same muscle. This shows that the inactivation of Ras within a few fibers stimulates autocrine-paracrine growth in the whole regenerating muscle. The amelioration of fresh weight and fiber size by dnRas declined after 21 days of regeneration, when the transfection was still detectable, suggesting a mechanism acting in the first half of muscle fiber growth.

TABLE 1
The increase of muscle mass and fiber size of regenerating soleus.
The values are in % of the mean mass (180 mg) and mean fiber size
(4261 μm²) ± SE (N = 3) of three normal soleus
muscles of rats corresponding with the age of the
animals with 28 days regenerating soleus.

| | Days of regeneration | | | |
|---|---|---|---|---|
| | 7 | 12 | 21 | 28 |
| Muscle weight | 50 ± 3.1 | 62 ± 2.4* | 79.4 ± 1.3** | 98 ± 4.8** |
| Fiber size | 25.9 ± 0.4 | 43.3 ± 0.65* | 58.4 ± 0.98** | 83.7 ± 1.4** |

*p < 0.05,
**p < 0.01 compared to the value of the previous stage.

### Example 2

### Inhibition of Calcineurin Activity Prevents the Growth Stimulation by dnRas

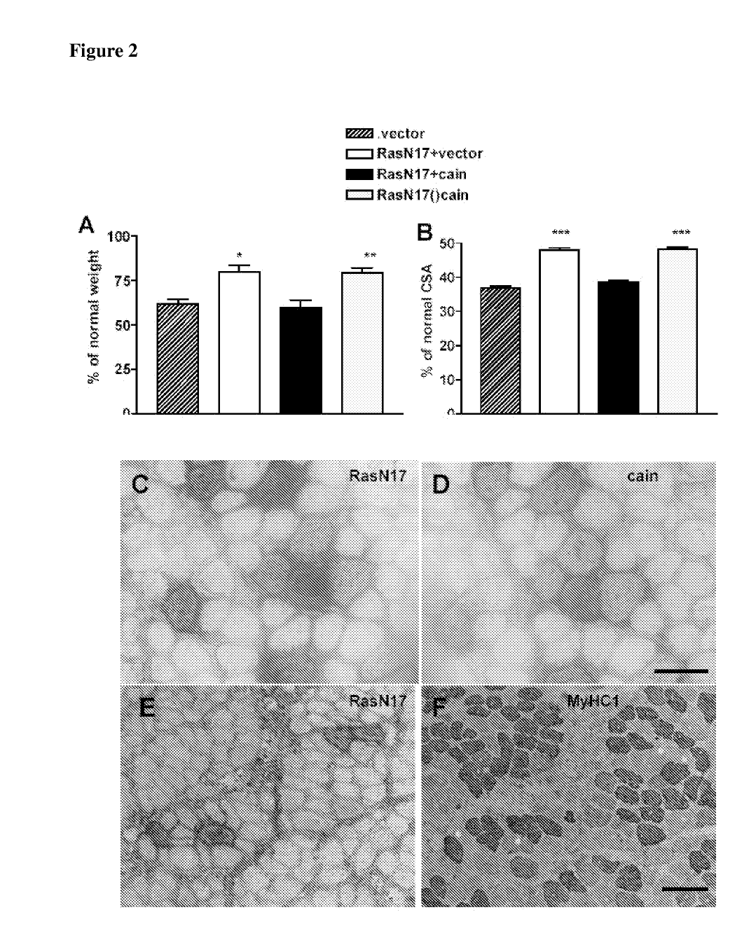

The calcineurin-NFAT-IL-4 is an important pathway in autocrine/paracrine fiber growth [8]. In order to check the connection between Ras and calcineurin, the regenerating soleus muscles were transfected with the mix of the dnRas expressing plasmid and a plasmid expressing the calcineurin inhibitor cain/cabin [19]. The transfection with such a plasmid mix practically results in only co-transfected fibers [5,21]. No difference was found in respect of fresh weight, muscle length and fiber size between the co-transfected muscles and the controls at day 12 of regeneration (FIG. 2AB) showing that the inhibition of calcineurin prevented the growth stimulation by dnRas. However, when the empty plasmid of cain was co-transfected with dnRas into the regenerating soleus muscles, the fresh weight and fiber size was increased similar to the muscles transfected only with dnRas (FIG. 2AB). This shows that the calcineurin activity is essential in the Ras-inactivated fibers for the autocrine-paracrine stimulation of fiber growth in the whole regenerating soleus. The separate transfection of dnRas and cain into different fibers of the same regenerating soleus muscles did not prevent the increase of fresh weight and fiber size (FIG. 2AB). In this case the cain transfected fibers were identified by the lack of expression of pan Ras and the myosin heavy chain 1 (MyHC1) (FIG. 2EF), since cain, similar to dnRas [11], prevents the expression of MyHC1 in the fibers of regenerating soleus muscle [20].

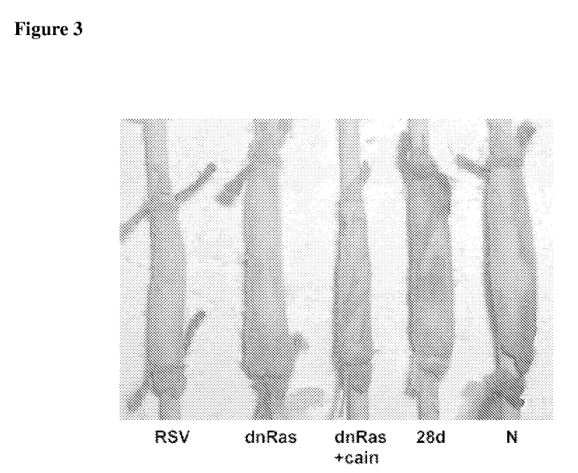

The dnRas transfected muscles also appeared larger in length than the dnRas+cain cotransfected muscles and the transfection controls (FIG. 3).

### Example 3

### Subcutaneous Injection of an IL-4 Specific Antibody Prevents Regenerating Fiber Growth

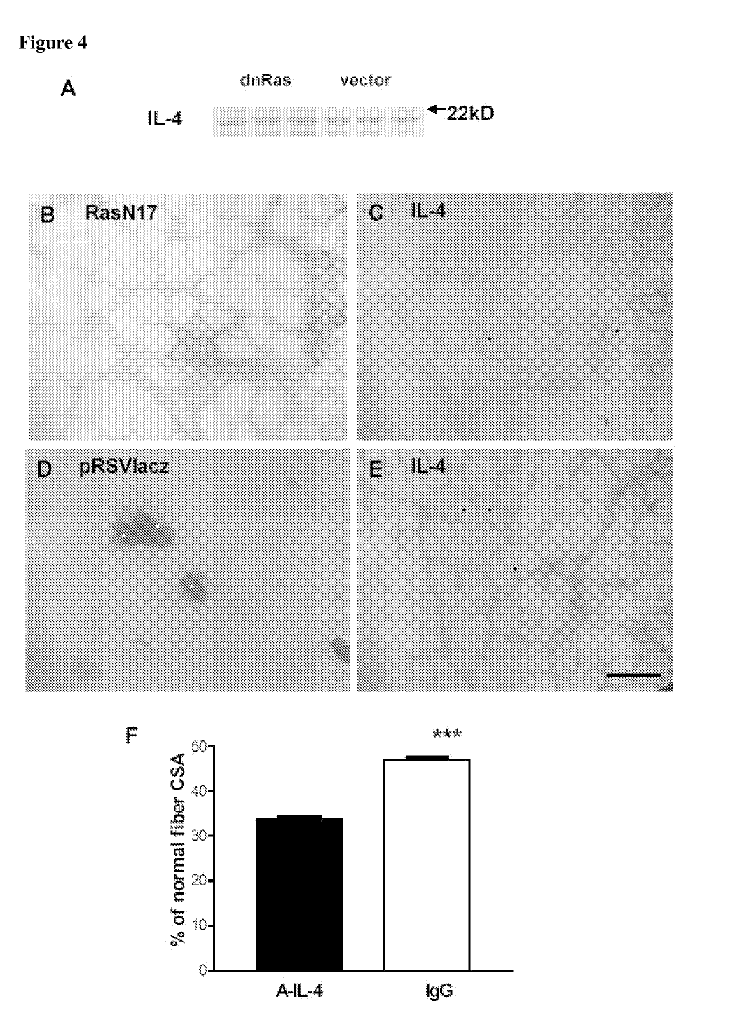

It has been reported that calcineurin-NFAT upregulates the expression of the interleukin-4 gene in developing muscle fibers [8]. The immunoblot did not show a difference in the IL-4 level of dnRas transfected regenerating so-lens muscles compared to the controls (FIG. 4A). However, the immunostaining for interleukin-4 showed a small increase of labeling around 15% of the dnRas expressing fibers compared to the utransfected fibers (FIG. 4BC). In contrast, in the pRSV lacz transfected control muscles the cytokine was not more detectable around the lacz expressing fibers than at the periphery of the untransfected ones (FIG. 4 DE). In order to test whether the increase of interleukin-4 is functionally involved in the calcineurin dependent growth stimulation by dnRas, a specific antibody for IL-4 was injected subcutaneously to the leg of the dnRas transfected regenerating soleus. This treatment prevented the fiber growth, while the injection of an irrelevant mouse IgG did not show any effect (FIG. 4F). This shows that the interleukin-4 is essential for the dnRas-ameliorated autocrine-paracrine fiber growth in the regenerating soleus.

### Example 4

### Neonatal SERCA1b is the Dominant SERCA1 Protein in Early Regenerating Soleus

The expression of the neonatal SERCA1b Ca2+-pump isoform, as assessed by Western blotting with an antiserum against the SERCA1b-specific octapeptide tail, was found to be very low in the fragmented SR of normal adult rat soleus muscle [55] (FIG. 1a). However, during regeneration of an injured rat soleus muscle, SERCA1b expression was found to resume on the third day and it reached a maximum on days 5-10, when its level was about 50-fold higher than in the normal adult muscles. At later stages of regeneration, it declined back to the level of the normal muscle. This shows that the SERCA1b protein largely followed its mRNA level [53].

I compared the ratio of SERCA1a and SERCA1b on day 7 and day 21 of regeneration (FIG. 1b,c). The signal on the immunoblots obtained with a pan-specific anti-SERCA1 (recognizes SERCA1a and SERCA1b) and that with the SERCA1b-specific antibody were of comparable intensity at day 7, while at day 21 of regeneration, the pan-specific signal was clearly more pronounced than the SERCA1b-specific one. This indicates that at day 7 most or all of the SERCA1 can be accounted for by the SERCA1b splice variant, but at day 21 more SERCA1a is expressed than SERCA1b.

### Example 5

### Silencing SERCA1b by Directed RNAi

To investigate whether SERCA1b plays a specific role in muscle regeneration, Irelied on RNAi silencing of its expression by injecting the regenerating muscle with an appropriate RNAi vector, also expressing GFP as a transfection reporter (see materials and methods). To check the silencing effect of the SERCA1b RNAi vector, it was first transfected into COS-1 cells together with a SERCA1b-expressing plasmid. The SERCA1b RNAi was found to knock down the mRNA level of SERCA1b to nearly 30% of that of a control transfected with the empty vector only (FIG. 2). This showed that the RNAi is a potent inhibitor of SERCA1b expression. Ialso included a mismatch control in which one nucleotide was deleted in the RNAi oligo (corresponding to position 3137 in the SERCA1b sequence). Apparently this mismatch sufficiently eliminated the SERCA1b silencing effect of the RNAi.

### Example 6

### SERCA1b-Directed RNAi Stimulates Muscle Regeneration

The SERCA1b RNAi vector was injected into a four-days regenerating muscle. At this stage of muscle regeneration myotubes and developing myofibers expressing the neonatal SERCA1b dominate over other cells [45, 46]. Three days later, on day 7 of regeneration the RNAi-treated muscle showed an increase in fresh weight (FIG. 3a) and in fiber size (cross-sectional area, CSA, FIG. 3b) compared to a regenerating muscle injected with the vector lacking the RNAi insert and containing only the open reading frame of the GFP reporter. This increased fresh weight and fiber size persisted at least till day 11 of regeneration. The mismatch control however, did not show the SERCA1b silencing and the growth-promoting effect on day eleven of regeneration (see FIG. 3ab).

Remarkably, inspection of the GFP-positive fibers indicated that on day seven of regeneration only a limited number, about 0.5%, of the fibers (i.e. 14 out of 2800) were successfully transfected. About half of the GFP-positive fibers showed reduced staining for SERCA1b compared to the neighboring fibers, suggesting the effective silencing of the Ca2+ pump (FIG. 3c,d). Furthermore, the same fibers also lost their reaction with the pan SERCA1 antibody, thus further confirming the absence of SERCA1b (FIG. 3e). The fibers transfected with the empty vector did express SERCA1b (FIG. 3f-h). Together these data show that the RNAi silencing of SERCA1b in only very few fibers exerted a remarkable widespread effect, such that it increased the fresh weight and fiber size in the entire muscle on day 11 of regeneration. Regaining the same fiber size would otherwise have required 21 days of regeneration. The precociously-regenerated RNAi-injected muscles showed a morphology similar to control regenerating muscle as judged by hematoxylin-eosin staining and slow myosin heavy chain (MyHC1) immunostaining (data not shown). The number of fibers per muscle cross section was found to remain constant between days 7 and 21 of regeneration both in normal [16] and in RNAi-treated muscles.

Most remarkable, therefore, is that the increase in fiber size was not restricted to those fibers that were successfully transfected nor to those in their immediate vicinity, but that it involved the whole muscle. Such an effect clearly suggests that an autocrine-paracrine process is involved.

### Example 7

### Coexpression of Cain with SERCA1b-Directed RNAi Prevents Stimulation of Fiber Growth

SERCA1b represents the most abundant SR Ca2+ pump in young fibers, therefore interfering with its expression might result in elevated sarcoplasmic Ca2+ and consequently in activation of calcineurin. The involvement of the calcineurin-NFAT-IL-4 pathway has been shown in autocrine-paracrine stimulation of fiber growth in regenerating muscle [56]. To check whether SERCA1b silencing might promote fiber growth through the elevation of calcineurin activity, Injected regenerating muscle with a 1:1 mix of the vector expressing the calcineurin binding fragment of the calcineurin inhibitor cain/cabin-1 [19] and the plasmid expressing SERCA1b RNAi. Under these conditions one can safely assume that the fibers will be simultaneously transfected by both plasmids and calcineurin and SERCA1b will be inhibited simultaneously in the same fibers [21, 56].

Coexpression of the calcineurin inhibitor was sufficient to suppress the effect of SERCA1b silencing on muscle fresh weight and fiber growth (FIG. 4a,b) showing that the calcineurin activity in the very same fiber in which the SERCA1b is silenced is essential in order to promote growth in the entire regenerating muscle. Indeed no such an inhibition was found when first SERCA1b RNAi was transfected and subsequently cain into the same muscle, a procedure that would result in the introduction of both cDNAs into different fibers. (FIG. 4a, b). As an indication of cotransfection Icompared the expression of GFP (reports the success of RNAi transfection) with the lack of expression of MyHC1 (reports cain effect). Indeed, cain is known to abolish the expression of MyHC1 [20] and, in case of cotransfection, the lack of MyHC1 also reported the expression of cain in the GFP-expressing fibers (FIG. 4c,d). However, when SERCA1b-GFP and cain were transfected one after the other, MyHC1 was not inhibited in the GFP-expressing fibers (FIG. 4e,f). It was possible to do these analyses at the earliest only from day 12, because this is the first day in regeneration when the MyHCI is clearly uniformly expressed in every fiber [15].

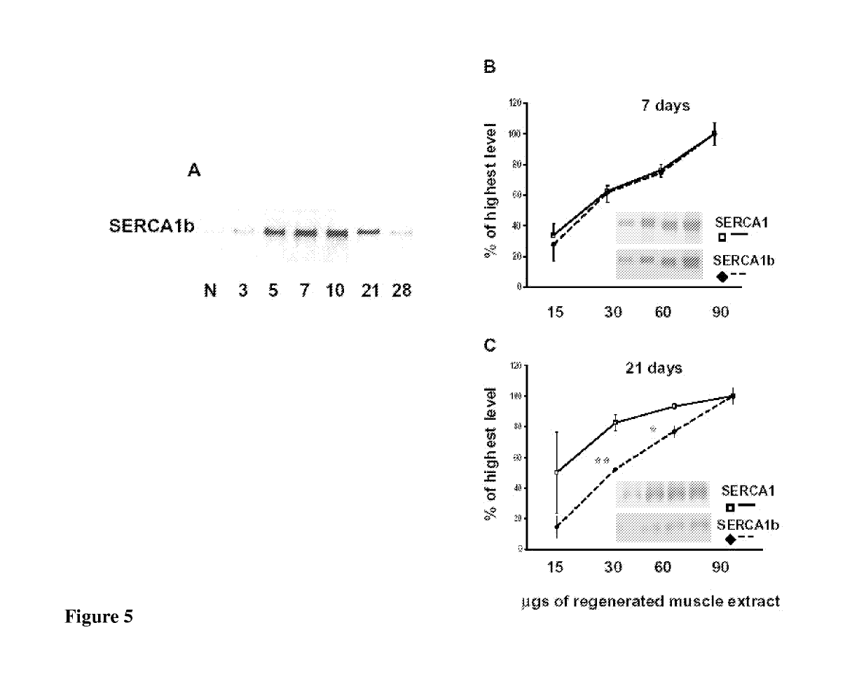

By the time of days 11-12 of regeneration the size, especially the length of the transfected muscles were clearly distinguishable as it is shown on FIG. 5.

### Example 8

### Interleukin-4 Might be a Mediator of SERCA1b-RNAi-Induced Fiber Growth

Muscle fiber growth is potentially controlled by a number of secreted factors [14], among these the effect of interleukin-4 has been demonstrated and it is found in regenerating muscle to rely on the calcineurin-NFAT pathway [56].

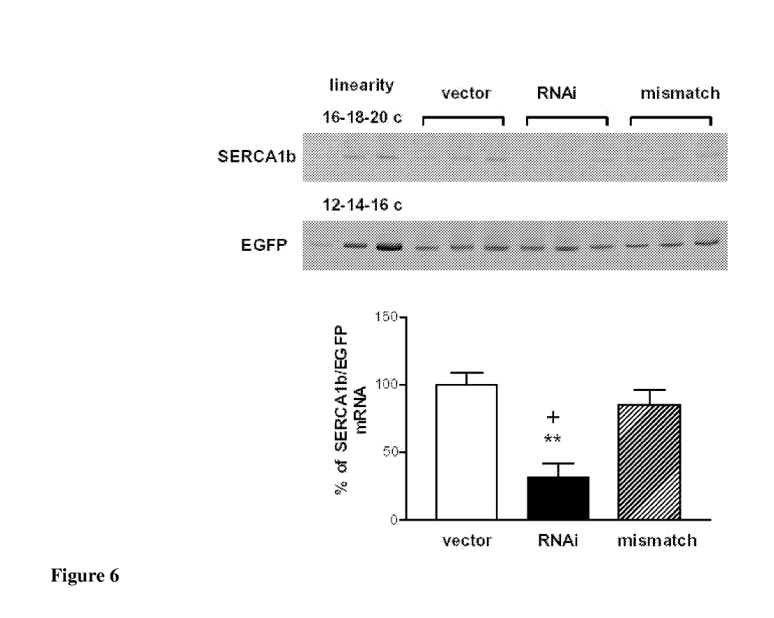

Interleukin-4 stimulates myoblast recruition to new myofibers, both in vitro and in vivo, which leads to an increase in fiber size and a higher number of myonuclei [8]. Therefore Icounted nuclei in regenerating muscles injected with the SERCA1b RNAi-expressing vector and found a higher number compared to muscles transfected with the empty vector or to the untransfected regenerating muscles (FIG. 6a).

The myonuclear number is reduced in interleukin-4 null mutant mice [8]. In an effort to interfere with the role of interleukin-4, Irepeatedly injected antiinterleukin-4 antibody in the vicinity of the SERCA1b-RNAi-transfected regenerating soleus muscle every day from day 7 until day 10 of regeneration. This injection prevented the SERCA1b-RNAi-induced fiber growth compared to the injection of an unrelated antibody. (FIG. 6b). As additional control, Ialso showed that there was no difference between the effects of anti-interleukin-4 and the unrelated antibody on the fiber size of regenerating muscles transfected with the empty RNAi vector. The expression of interleukin-4 was documented by immunoblotting in our SERCA1b silenced regenerating muscles (FIG. 6c). This suggests that the interleukin-4 is necessary as an autocrine-paracrine mediator of SERCA1b-RNAi-induced fiber growth in the whole regenerating soleus.

Discussion of the Experimental Findings

The inactivation of Ras in a few muscle fibers remarkably stimulates muscle mass and fiber growth in the entire regenerating soleus of the rat. The co-transfection with cain prevented the amelioration of growth showing that the calcineurin activity is essential in the dnRas transfected fibers for the stimulation of autocrine-paracrine fiber growth of the whole regenerating muscle. Such an effect was not observed when dnRas and cain/cabin were separately injected, achieving the transfection of different fibers of the same regenerating muscle. This showed that dnRas affected calcineurin within the same muscle fiber. Therefore the autocrine-paracrine growth stimulation is a down-stream effect of calcineurin. The calcineurin-NFAT pathway is known to induce the interleukin-4 gene and the cytokine stimulates the accretion of myoblasts into the fibers, increasing muscle growth [8]. The present inventor has hereby found that the injection of IL-4 antibody prevented the autocrine-paracrine fiber growth stimulation by dnRas. This suggests that dnRas increases the IL-4 expression in the transfected fibers by upregulating the calcineurin-NFAT-IL-4 pathway. The secreted IL-4 probably triggers the stimulation of autocrine-paracrine fiber growth in the whole muscle. Normally, Ras might oppose the activity of calcineurin in the regenerating muscle fibers but the inactivation of Ras releases the inhibition of calcineurin that is able to dephosphorylate NFAT. The dephosphorylated NFAT is imported into the nucleus therefore inducing the transcription of the IL-4 gene and ameliorating muscle fiber growth [8]. The calcineurin-opposing effect of Ras has been reported in T-lymphocytes when the Ras-dependent kinases GSK-3, CK1 and JNK stimulated the phosphorilation and the conclusive nuclear export of NFAT [22,23,24]. No similar mechanism has been reported in muscle cells.

Another possibility for the cross-talk between Ras and calcineurin, that Ras, similar to calcineurin-NFAT, has been shown to be dependent on the level of Ca²⁺ in cultured myotubes [6,25]. If Ras and calcineurin share the same Ca²⁺ signals it might be that the inactivation of Ras increases the availability of Ca²⁺ for calcineurin and upregulates the calcineurin-NFAT pathway.

It is important to note that the parallel inactivation of Ras and calcineurin in the same fibers did not decrease the size of the co-transfected fibers compared to the controls. This suggests that the concurrent inactivity of calcineurin and Ras in a few fibers is not interfering with normal growth. Instead, it is likely that the balanced activity of Ras and calcineurin within the fibers controls the autocrine-paracrine regulation of fiber size during the first half of soleus muscle regeneration.

I have established that the dnRas transfected fibers produced only slightly more interleukin-4 than the untransfected fibers. No difference in IL-4 levels was found on immunoblots between the dnRas transfected and the control transfected regenerating muscles. This implies that dnRas resulted in a small increase of the cytokine. The injection of the IL-4 antibody prevented the augmentation of muscle fiber growth but it did not decrease the fiber size below the control level. This shows that secretion of IL-4 is more required for the stimulation than for the normal growth. It has been reported that calcineurin mediates the downstream effect of IGF-1 [26-28]. This implies that besides IL-4, other factors might also be involved in the augmentation of autocrine-paracrine fiber growth. Accordingly, potentially secretable factors (secretomes) are found in a great number in human skeletal muscle libraries [14].

The present findings have a clear and definite implication for the molecular therapy of muscle dystrophies. It is a recently developed strategy that the whole muscle size is increased by transfecting a portion of muscle fibers with plasmids expressing secretable factors like IGF-I and IL-4 [29]. The present invention provides a novel approach for stimulating the endogenous autocrine-paracrine muscle growth via manipulation of intrinsic pathways. This would require the transfection of only a few muscle fibers and might open an interesting perspective in the treatment of muscle injuries and degenerative-regenerative muscular disorders. The gene therapy in muscle is hampered by the low transfection efficiency but the fiber growth stimulation enabled by the present invention overcomes this restrain. A person skilled in the pertinent art will easily comprehend that this treatment is most probably applicable also to other muscles than the soleus. The calcineurin-NFAT pathway is generally found in developing muscle fibers [8], its level [10] and effect [30] are dependent on slow type innervation and variable in the phenotypically different muscles [31,32]. However a recent report shows that activation of one isoform of the active subunit of calcineurin, calcineurin-Aα enhances regeneration of the tibialis anterior, a predominantly fast muscle in mouse [33]. Therefore it is quite probable that calcineurin is essential in the regeneration of phenotypically different muscles.

My further findings demonstrate that SERCA1b is also an important protein for muscle fiber development. In vivo silencing of this Ca²⁺ pump in about 0.5% of the fibers of a regenerating soleus, increased the fiber size in the entire muscle. Upon this treatment the muscle regained its normal dimensions in a week, instead of in 17 days like the controls. Apparently, this effect involved autocrine-paracrine pathways resembling to the effect of dnRas [56]. Therefore Ifelt prompted to investigate the intrinsic role of calcineurin in the SERCA1b RNAi transfected fibers. The cotransfection of a calcineurin inhibitor cain/cabin-1 with the SERCA1b-silencing vector showed that the intrinsic calcineurin activity is needed for the SERCA1b-silenced fibers to promote growth in the entire regenerating muscle. Elements of the calcineurin-NFAT pathway have already been connected to muscle differentiation in in vitro experiments. The transfection with a viral vector overexpressing cain inhibited myogenesis of L6 muscle cells [39]. Inhibition of all SERCA pumps by thapsigargin in cultured muscle cells increased the activity of an NFAT reporter construct [34]. The NFATC2−/− mice exhibited normal muscle formation after injury but displayed impaired myofiber growth with reduced cross sectional area compared to normal [8]. However our work is the first to show that in vivo SERCA1b is required for controlling fiber growth in regenerating muscle. An earlier report that inhibition of SERCA by thapsigargin in cultured muscle cells decreased myotube growth and differentiation [49, 50] seems contradictory to the stimulatory effect of SERCA1b RNAi transfection on muscle growth that I have demonstrated hereby. However it should be taken into consideration that there are numerous differences between the in vitro and in vivo condition (e.g. the absence or presence of innervation). Silencing of SERCA1b in only a few fibers, as opposed to in the whole regenerating muscle, is probably a major prerequisite for growth stimulation. Fibers with silenced SERCA1b activate gene expression via the calcineurin-NFAT pathway and secrete signals to the rest of the regenerating muscle. This probably triggers a growth stimulating compensatory response in the nontransfected fibers. The lack of difference in size of transfected and untransfected fibers testifies for a very efficient performance of the autocrine/paracrine network in SERCA1b RNAi stimulated regeneration.

Recently interleukin-4 has been reported to act as a molecular signal supporting muscle fiber growth. This cytokine is secreted by a subset of nascent myotubes and stimulates myoblast accretion by acting on its receptor expressed on the surface of all myoblasts [8]. The expression of interleukin-4 is dependent on transcription factor NFATc2 regulated by calcineurin in an early stage of myofiber growth [34, 6, 8]. Therefore, when Isilenced SERCA1b, the sarcoplasmic Ca²⁺ level was increased and might have enhanced the calcineurin-NFAT pathway. In view of this finding SERCA1b and calcineurin mediate the NFATc2-IL4—IL-4R autocrine-paracrine pathway which is governing accretion of myoblasts by the developing muscle fibers. However, interfering both with SERCA1b expression and calcineurin activity in the same fibers stops this response. Though not intending to be bound to any theoretical mechanism of action, I hypothesize that interleukin-4 as an NFAT inducible cytokine in muscle [8] might be an important factor of this compensatory response. The increased myonuclear number of SERCA1b RNAi treated muscle was in agreement with this suggestion and the effect of perimuscular injection of monoclonal antibody confirmed the involvement of interleukin-4 in the SERCA1b RNAi stimulated growth. In the same set of experiments the regeneration of muscles injected with an empty RNAi vector was not influenced by antiinterleukin-4 and this suggested a lower importance of the cytokine in normal regeneration than in the SERCA1b silenced muscle growth. However, null mutant mice for the IL-4 or the IL-4 receptor a subunit display impaired fiber growth in regeneration after injury [8]. Therefore the failure of gaining response to the anti-IL-4 treatment in regenerating soleus injected with the empty vector might be because of the limitations of the applied methodology, i.e. concentration of the antibody. In any case, this suggests, that it is of interest to carry out further experiments on investigating the nature of Il-4 influence on intact muscle regeneration.

Remarkably, the intervention in accordance with the invention did not result in additional hypertrophy above the normal muscle size, in spite of the fact that the expression of the GFP from the RNAi vector was still detectable 17 days after the injection (day 21 of regeneration). In contrast, the silencing effect of RNAi on SERCA1b (as judged by the immunostaining) was already lost 3 days after the injection, i.e. at day 7 of regeneration. This shows that SERCA1b controls the size of the developing muscle fibers but not that of the mature fibers. The stimulating effect of SERCA1b RNAi on the regenerating muscle was strikingly similar to the effect of dnRas (as detailed above), therefore it is likely that in the regenerating soleus both Ras and SERCA1b are upstream controllers of calcineurin activity. However the mechanism of inhibition of the calcineurin-NFAT-IL-4 pathway seems to be more direct by SERCA1b than by Ras. This might be also indicated by the increased myonuclear number in response to SERCA1b inhibition but not to the transfection with dnRas. In conclusion, the neonatal SERCA1b and calcineurin are pivotal elements of the Ca²⁺ signaling that controls autocrine/paracrine muscle growth via interleukin-4 in development but, very importantly, is apparently not involved in hypertrophy of adult skeletal muscles. These findings clearly enable the development of novel treatment methods for enhancing fiber growth in regenerating skeletal muscles.

On the basis of the above discussion, the person skilled in the pertinent art will fully comprehend that even though the very important inventive concept of the present invention, namely that upregulation of the calcineurin-NFAT-IL-4 pathway via upstream controllers enhances fiber growth in regenerating skeletal muscles without causing any hypertrophy, was experimentally only demonstrated for two different upstream controller (namely the expression of the Ras and SERCA1b gene products) and by two specific silencer (namely the expression of dnRas and SERCA1b RNAi), similar effect may be most probably reached by other active agents capable of upregulating the calcineurin-NFAT-IL-4 pathway. Such useful active agents may be exemplified by different SERCA inhibiting agents that are capable of inducing capacitive calcium entry (CCE) via the opening of the so called store-operated channels (SOCs), whereby the elevated local Ca²⁺ level upregulates the calcineurin pathway [58, 59].

Substances capable of upregulating the calcineurin-NFAT-IL-4 pathway by inhibiting the activity of the Ras or SERCA1b gene product may be capable of said upregulation e.g. by inhibiting the expression of said gene products or by inhibiting the activity of the expressed gene products. Such advantageous substances are e.g. vectors enabling the expression of RNA or protein molecules capable of inhibiting the expression of the Ras or SERCA1b gene or the activity of the Ras or SERCA1b gene product in skeletal muscle cells. Such advantageous active agents that are useful in accordance with the present invention are e.g. vectors encoding dominant negative Ras (dnRas) or vectors capable of expressing a SERCA1b-RNAi as exemplified above. The person skilled in the pertinent art will, however, fully comprehend that the calcineurin-NFAT-IL-4 pathway may be triggered via other routes and by using other substances than those specifically exemplified herein. Such other substances may be easily identified by a person skilled in the pertinent art without undue burden on the basis of the present disclosure and, therefore, such substances are considered as being covered by the scope of the present invention.

### REFERENCES

[1] R. G. Whalen, J. B. Harris, G. S. Butler-Browne, S. Sesodia, Expression of myosin isoforms during notexin-induced regeneration of rat soleus muscles, Dev. Biol. 141 (1990) 24-40.
[2] R. Bassel-Duby, E. N. Olson, Signaling pathways in skeletal muscle remodeling, Annu. Rev. Biochem. 75 (2006) 19-37.
[3] M. A. Carrasco, C. Hidalgo, Calcium microdomains and gene expression in neurons and skeletal muscle cells, Cell Calcium 40 (2006) 575-583.
[4] K. J. McCullagh, E. Calabria, G. Pallafacchina, S. Ciciliot, A. L. Serrano, C. Argentini, J. M. Kalhovde, T. Lomo, S. Schiaffino, NFAT is a nerve activity sensor in skeletal muscle and controls activity-dependent myosin switching, Proc. Natl. Acad. Sci. U.S.A., 101 (2004) 10590-10595.
[5] J. Tothova, B. Blaauw, G. Pallafacchina, R. Rudolf, C. Argentini, C. Reggiani, S. Schiaffino, NFATc1 nucleocytoplasmic shuttling is controlled by nerve activity in skeletal muscle, J. Cell Sci. 119 (2006) 1604-1611.
[6] V. Horsley, B. B. Friday, S. Matteson, K. M. Kegley, J. Gephart, G. K. Pavlath, Regulation of the growth of multinucleated muscle cells by an NFATC2-dependent pathway, J. Cell Biol. 153 (2001) 329-338.
[7] G. K. Pavlath, V. Horsley, Cell fusion in skeletal muscle—central role of NFATC2 in regulating muscle cell size, Cell Cycle 2 (2003) 420-423.
[8] V. Horsley, K. M. Jansen, S. T. Mills, G. K. Pavlath, IL-4 acts as a myoblast recruitment factor during mammalian muscle growth, Cell 113 (2003) 483-494.
[9] S. E. Dunn, A. R. Simard, R. Bassel-Duby, R. S. Williams, R. N. Michel, Nerve activity-dependent modulation of calcineurin signaling in adult fast and slow skeletal muscle fibers, J. Biol. Chem. 276 (2001) 45243-45254.
[10] R. Fenyvesi, G. Racz, F. Wuytack, E. Zador, The calcineurin activity and MCIP1.4 mRNA levels are increased by innervation in regenerating soleus muscle, Biochem. Biophys. Res. Commun. 320 (2004) 599-605.
[11] M. Murgia, A. L. Serrano, E. Calabria, G. Pallafacchina, T. Lomo, S. Schiaffino, Ras is involved in nerve-activity-dependent regulation of muscle genes, Nat. Cell Biol. 2 (2000) 142-147.
[12] S. C. Bodine, T. N. Stitt, M. Gonzalez, W. O. Kline, G. L. Stover, R. Bauerlein, E. Zlotchenko, A. Scrimgeour, J. C. Lawrence, D. J. Glass, G. D. Yancopoulos, Akt/mTOR pathway is a crucial regulator of skeletal muscle hypertrophy and can prevent muscle atrophy in vivo, Nat. Cell Biol. 11 (2001) 1014-1019.
[13] G. Pallafacchina, E. Calabria, A L. Serrano, J. M. Kalhovde, S. Schiaffino, A protein kinase B-dependent and rapamycin-sensitive pathway controls skeletal muscle growth but not fiber type specification, Proc. Natl. Acad. Sci. U.S.A. 99 (2002) 9213-9218.
[14] S. Bortoluzzi, P. Scannapieco, A. Cestaro, G. A. Danieli, S. Schiaffino, Computational reconstruction of the human skeletal muscle secretome, Proteins. 62 (2006) 776-792.
[15] E. Zador, F. Wuytack, Expression of SERCA2a is independent of innervation in regenerating soleus muscle, Am. J. Physiol. Cell Physiol. 285 (2003) C853-861.
[16] E. Zador, R. Fenyvesi, F. Wuytack, Expression of SERCA2a is not regulated by calcineurin or upon mechanical unloading in skeletal muscle regeneration, FEB S Lett. 579 (2005) 749-752.
[17] M. B. Ramocki, S. E. Johnson, M. A. White, C. L. Ashendel, S. F. Konieczny, E. J. Taparowsky, Signaling through mitogen-activated protein kinase and Rac/Rho does not duplicate the effects of activated Ras on skeletal myogenesis, Mol. Cell. Biol. 17 (1997) 3547-3555.
[18] M. B. Ramocki, M. A. White, S. F. Konieczny, E. J. Taparowsky, A role for RalGDS and a novel Ras effector in the Ras-mediated inhibition of skeletal myogenesis, J. Biol. Chem. 273 (1998) 17696-17701.
[19] M. M. Lai, P. E. Burnett, H. Wolosker, S. Blackshaw, S. H. Snyder, Cain, a novel physiologic protein inhibitor of calcineurin, J. Biol. Chem. 273 (1998) 18325-18331.
[20] A. L. Serrano, M. Murgia, G. Pallafacchina, E. Calabria, P. Coniglio, T. Lømo, S. Schiaffino, Calcineurin controls nerve activity-dependent specification of slow skeletal muscle fibers but not muscle growth, Proc. Natl. Acad Sci USA. 98 (2001) 13108-13113.
[21] Z. A. Rana, M. Ekmark, K Gundersen, Coexpression after electroporation of plasmid mixtures into muscle in vivo, Acta Physiol Scand. 181 (2004) 233-238.
[22] C. W. Chow, M. Rincon, J. Cavanagh, M. Dickens, R. J. Davis, Nuclear accumulation of NFAT4 opposed by the JNK signal transduction pathway, Science 278 (1997) 1638-1641.
[23] C. R. Beals, C. M. Sheridan, C. W. Turck, P. Gardner, G. R. Crabtree, Nuclear export of NF-ATc enhanced by glycogen synthase kinase-3, Science 275 (1997) 1930-1934.
[24] H. Okamura, C. Garcia-Rodriguez, H. Martinson, J. Qin, D. M. Virshup, A. Rao, A conserved docking motif for CK1 binding controls the nuclear localization of NFAT1, Mol. Cell. Biol. 24 (2004) 4184-4195.
[25] M. Estrada, A. Espinosa, M. Muller, E. Jaimovich, Testosterone stimulates intracellular calcium release and mitogen-activated protein kinases via a G protein-coupled receptor in skeletal muscle cells, Endocrinology 144 (2003) 3586-3597.
[26] A. Musaro, K. J. McCullagh, F. J. Naya, E. N. Olson, N. Rosenthal, IGF-1 induces skeletal myocyte hypertrophy through calcineurin in association with GATA-2 and NFATc1, Nature 400 (1999) 581-585.
[27] C. Semsarian, M. J. Wu, Y. K. Ju, T. Marciniec, T. Yeoh, D. G. Allen, R. P. Harvey, R. M. Graham, Skeletal muscle hypertrophy is mediated by a Ca2+-dependent calcineurin signalling pathway, Nature 400 (1999) 576-81.
[28] D. R. Vyas, E. E. Spangenburg, T. W. Abraha, T. E. Childs, F. W. Booth, GSK-3beta negatively regulates skeletal myotube hypertrophy, Am. J. Physiol. Cell Physiol. 283, (2002) C545-551.
[29] J. D. Schertzer, D. R. Plant, G. S. Lynch, Optimizing plasmid-based gene transfer for investigating skeletal muscle structure and function, Mol. Ther. 13 (2006) 795-803.
[30] T. Launay, P. Noirez, G. Butler-Browne, O. Agbulut, Expression of slow myosin heavy chain during muscle regeneration is not always dependent on muscle innervation and calcineurin phosphatase activity, Am. J. Physiol. Regul. Integr. Comp. Physiol. 290 (2006) R1508-1514.
[31] P. O. Mitchell, S. T. Mills, G. K. Pavlath, Calcineurin differentially regulates maintenance and growth of phenotypically distinct muscles, Am. J. Physiol. Cell Physiol. 282 (2002) C984-992.
[32] R. J. Talmadge, J. S. Otis, M. R. Rittler, N. D. Garcia, S. R. Spencer, S. J. Lees, F. J. Naya, Calcineurin activation influences muscle phenotype in a muscle-specific fashion, BMC Cell. Biol. 5 (2004) 28.
[33] N. Stupka, J. D. Schertzer, R. Bassel-Duby, E. N. Olson, G. S. Lynch, Calcineurin-A alpha activation enhances the structure and function of regenerating muscles after myotoxic injury, Am. J. Physiol. Regul. Integr. Comp. Physiol. 293 (2007) R686-694.
[34] Abbott K L, Friday B B, Thaloor D, Murphy T J & Pavlath G K (1998). Activation and cellular localization of the cyclosporine A-sensitive transcription factor NF-AT in skeletal muscle cells. Mol Biol Cell 9, 2905-2916.
[35] Brandi C J, DeLeon S, Martin D R & MacLennan D H (1987). Adult forms of the Ca2+ ATPase of sarcoplasmic reticulum. Expression in developing skeletal muscle. J Biol Chem 262, 3768-3774.
[36] Brandi C J, Green N M, Korczak B & MacLennan D H (1986). Two Ca2+ ATPase genes: homologies and mechanistic implications of deduced amino acid sequences. Cell 44, 597-607.
[37] Brody I A (1969). Muscle contracture induced by exercise. A syndrome attributable to decreased relaxing factor. N Engl J Med 281, 187-192.
[38] Brummelkamp T R, Bernards R & Agami R (2002). A system for stable expression of short interfering RNAs in mammalian cells. Science 296, 550-553.
[39] Friday B B, Horsley V & Pavlath G K (2000). Calcineurin activity is required for the initiation of skeletal muscle differentiation. J Cell Biol 149, 657-665.
[40] Gauthier E R, Madison S P & Michel R N (1997). Rapid RNA isolation without the use of commercial kits: application to small tissue samples. Pflügers Arch Eur J Physiol 433, 664-668.
[41] Harris J B, Johnson M A & Karlsson E (1975). Pathological responses of rat skeletal muscle to a single subcutaneous injection of a toxin isolated from the venom of the Australian tiger snake, Notechis scutatus scutatus. Clin Exp Pharmacol Physiol 2, 383-404.
[42] Horsley V & Pavlath G K (2004). Forming a multinucleated cell: molecules that regulate myoblast fusion. Cells Tissues Organs 176, 67-78.
[43] Korczak B, Zarain-Herzberg A, Brandi C J, Ingles C J, Green M N & MacLennan D H (1988). Structure of the rabbit fast-twitch skeletal muscle Ca2+ ATPase gene. J Biol Chem 263, 4813-4819.
[44] Maruyama K & MacLennan DH (1988). Mutation of aspartic acid-351, lysine-352, and lysine-515 alters the Ca2+ transport activity of the Ca2+-ATPase expressed in COS-1 cells. Proc Natl Acad Sci USA 85, 3314-3318.
[45] Mendler L, Szakonyi G, Zador E, Gorbe A, Dux L & Wuytack F (1998). Expression of sarcoplasmic/endoplasmic reticulum Ca2+ ATPases in rat extensor digitorum longus (EDL) muscle regenerating from notexin-induced necrosis. J Muscle Res Cell Motil 19, 777-785.
[46] Mendler L, Zador E, Dux L & Wuytack F (1998). mRNA levels of myogenic regulatory factors in rat slow and fast muscles regenerating from notexin-induced necrosis. Neuromusc Disorders 8, 533-541.
[47] Moss F P & Leblond C P (1971). Satellite cells as the source of nuclei in muscles of growing rats. Anat Rec 170, 421-35.
[48] Pan Y, Zvaritch E, Tupling R W, Rice J, de Leon S, Rudnicki M, McKerlie C, Banwell B L & MacLennan D H (2003). Targeted disruption of the ATP2A1 gene encoding the sarco(endo)plasmic reticulum Ca2+ ATPase isoform 1 (SERCA1) impairs diaphragm function and is lethal in neonatal mice. J Biol Chem 278, 13367-13375.
[49] Porter G A Jr, Makuck R F & Rivkees A A (2002). Reduction in intracellular calcium levels inhibits myoblast differentiation. J Biol Chem 277, 28942-28947.
[50] Seigneurin-Venin S, Parrish E, Marty I, Rieger F, Romey G, Villaz M & Garcia L (1996). Involvement of the dihydropyridine receptor and internal Ca2+ stores in myoblast fusion. Exp Cell Res 223, 301-307.
[51] Zador E, Bottka S & Wuytack F (2002). Antisense inhibition of myoD expression in regenerating rat soleus muscle is followed by an increase in the mRNA levels of myoD, myf-5 and myogenin and by a retarded regeneration. BBA Mol Cell Res 1590, 52-63.
[52] Zador E, Mendler L, Talthes, V, De Bleecker J & Wuytack F (2001). Regenerating soleus and extensor digitorum longus muscles of the rat show elevated levels of TNF-alpha and its receptors, TNFR-60 and TNFR-80. Muscle Nerve 24, 1058-1067.
[53] Zador E, Mendler L, Ver Heyen M, Dux L & Wuytack F (1996). Changes in mRNA levels of the sarcoplasmic-reticulum Ca2+ ATPase isoforms in the rat soleus muscle regenerating from notexin-induced necrosis. Biochem J 320, 461-464.
[54] Zador E, Szakonyi G, Racz G, Mendler L, Ver Heyen M, Lebacq J, Dux L & Wuytack F (1998). Expression of sarcoplasmic/endoplasmic reticulum Ca2+ ATPases in the rat extensor digitorum longus (EDL) muscle regenerating from notexin-induced necrosis. Acta Histochem 100, 355-369.
[55] Zador E, Vangheluwe P & Wuytack F (2007). The expression of the neonatal sarcoplasmic reticulum Ca(2+) pump (SERCA1b) hints to a role in muscle growth and development. Cell Calcium 41, 379-88.
[56] Zador E (2008). dnRas stimulates autocrine-paracrine growth of regenerating muscle via calcineurin-NFAT-IL-4 pathway. Biochem Biophys Res Com 375, 265-70.
[57] Zubrzycka-Gaarn E, MacDonald G, Phillips L, Jorgensen A O & MacLennan D H (1984). Monoclonal anti-bodies to the Ca2++Mg2+-dependent ATPase of sarcoplasmic reticulum identify polymorphic forms of the enzyme and indicate the presence in the enzyme of a classical high-affinity Ca2+ binding site. J Bioenergetics Biomembranes 16, 441-464.
[58] Rosenberg P, Hawkins A, Stiber J, Shelton J M, Hutcheson K, Bassel-Duby R, Shin D M, Yan Z, Williams R S. (2004) TRPC3 channels confer cellular memory of recent neuromuscular activity. Proc Natl Acad Sci USA. 101:9387-92.
[59] Vazquez G, Lievremont J P, St J Bird G, Putney J W Jr. (2001) Human Trp3 forms both inositol trisphosphate receptor-dependent and receptor-independent store-operated cation channels in DT40 avian B lymphocytes. Proc Natl Acad Sci USA. 98(20):11777-82

1. A substance capable of upregulating the calcineurin-NFAT-IL-4 pathway in an organism having skeletal muscle for use in facilitating the growth or regeneration of skeletal muscle in said organism.

2. The substance according to claim 1, for use in the treatment or prevention of degenerative-regenerative muscular disorders or in the treatment of muscle injuries.

3. Use of a substance capable of upregulating the calcineurin-NFAT-IL-4 pathway in an organism having skeletal muscle for the preparation of a pharmaceutical composition for the treatment or prevention of degenerative-regenerative muscular disorders, facilitating the growth or regeneration of skeletal muscle or for the treatment of muscle injuries in said organism.

4. A substance or use according to any of claims 1-3, wherein said substance is capable of upregulating the calcineurin-NFAT-IL-4 pathway by inhibiting the activity of the Ras or SERCA1b gene product.

5. A substance or use according to claim 4, wherein said substance is a vector enabling the expression of an RNA or protein molecule capable of inhibiting the expression of the Ras or SERCA1b gene or the activity of the Ras or SERCA1b gene product in skeletal muscle cells, advantageously a vector encoding dominant negative Ras (dnRas) or a vector capable of expressing a SERCA1b-RNAi.

6. A substance or use according to any of claims 1-5, wherein said treatment includes gene therapy.

7. A substance or use according to any of claims 1-6, wherein said treatment is made by introducing said substance only into a part of the muscle fibers present in a skeletal muscle to be treated, advantageously into about 0.1-10%, about 0.2-5%, about 0.5-2% or about 1% of the muscle fibers present in said skeletal muscle, whereby said growth or regeneration facilitating effect induced will be detectable both in fibers of said muscle where said substance was introduced and in fibers where it was not.

8. A substance or use according to any of claims 1-7, wherein said organism is a vertebrate, advantageously a mammal and more advantageously human.

9. A method for identifying a substance useful for facilitating the growth or regeneration of skeletal muscle, or the treatment or prevention of degenerative-regenerative muscular disorders, or the treatment of muscle injuries, comprising the steps of
i) providing a cell or tissue culture or a model animal in which the activity of calcineurin is experimentally assessable;
ii) contacting a test substance with said cell or tissue culture or model animal;
iii) assessing the activity of calcineurin in said cell or tissue culture or model animal after contacting the same with said test substance; and
iv) identifying said test substance as a substance useful in the treatment or prevention of degenerative-regenerative muscular disorders or in the treatment of muscle injuries if the calcineurin activity assessed in step iii) is significantly higher than the calcineurin activity assessable in said cell or tissue culture or model animal before said contacting with said test substance performed in step ii).

10. A method for identifying a substance useful for facilitating the growth or regeneration of skeletal muscle, or the treatment or prevention of degenerative-regenerative muscular disorders, or the treatment of muscle injuries, comprising the steps of
i) providing a cell or tissue culture or a model animal in which the activity of the Ras or SERCA1b gene is experimentally assessable;
ii) contacting a test substance with said cell or tissue culture or model animal;
iii) assessing the activity of the Ras or SERCA1b gene in said cell or tissue culture or model animal after contacting the same with said test substance; and
iv) identifying said test substance as a substance useful in the treatment or prevention of degenerative-regenerative muscular disorders or in the treatment of muscle injuries if the Ras or SERCA1b activity assessed in step iii) is significantly lower than the Ras or SERCA1b activity assessable in said cell or tissue culture or model animal before said contacting with said test substance performed in step ii).

11. A method for facilitating the growth or regeneration of skeletal muscle, or treating or preventing degenerative-regenerative muscular disorders, or treating muscle injuries comprising introducing a substance capable of upregulating the calcineurin-NFAT-IL-4 pathway into said skeletal muscle.

12. The method of claim 11, wherein said substance is a substance which is capable of upregulating the calcineurin-NFAT-IL-4 pathway by inhibiting the activity of the Ras or SERCA1b gene product.

13. The method of claim 12, wherein said introduction is made only into a part of the muscle fibers present in a skeletal muscle to be treated, advantageously into about 0.1-10%, about 0.2-5%, about 0.5-2% or about 1% of the muscle fibers present in said skeletal muscle, whereby said growth or regeneration facilitating effect induced will be detectable both in fibers of said muscle where said substance was introduced and in fibers where it was not.

14. The method of any of claims 10-13, wherein said substance is a substance capable of inhibiting the activity of the Ras or SERCA1b gene, advantageously a vector enabling the expression of an RNA or protein molecule capable of inhibiting the expression of the Ras or SERCA1b gene or the activity of the Ras or SERCA1b gene product in skeletal muscle cells, more advantageously a vector encoding dominant negative Ras (dnRas) or a vector capable of expressing a SERCA1b-RNAi.
